(19) United States
(12) Patent Application Publication
(10) Pub. No.: US 2025/0163373 A1
(43) Pub. Date: May 22, 2025

(54) AN INNERVATED CELLULAR COMPOSITE

(71) Applicant: ALCYOMICS LTD, Newcastle upon Tyne (GB)

(72) Inventors: Anne DICKINSON, Newcastle upon Tyne (GB); Matthew FREER, Newcastle upon Tyne (GB)

(21) Appl. No.: 18/833,318

(22) PCT Filed: Jan. 31, 2023
(86) PCT No.: PCT/GB2023/050212

§ 371 Date: Jul. 25, 2024

(30) Foreign Application Priority Data
Jan. 31, 2022 (GB) 2201229.8

(51) Int. Cl. C12N 5/071; C12N 5/077; C12N 5/079; C12N 5/0793

(52) U.S. Cl. CPC C12N 5/0629; C12N 5/0619; C12N 5/0622; C12N 5/0656; C12N 2513/00

(57) ABSTRACT

The present invention relates to a cellular composite, which advantageously may be innervated. The invention also relates to methods of making said cellular composite, uses of said cellular composite, and screening methods utilising said cellular composite and uses of the cellular composite.

### FIELD OF THE INVENTION

The present invention relates to a cellular composite, which advantageously may be innervated. The invention also relates to methods of making said cellular composite, uses of said cellular composite, and screening methods utilising said cellular composite.

### BACKGROUND

Neurotoxicity is a major side effect of many chemotherapeutic treatment regimes with an estimated 60% of patients undergoing some form of neurotoxicity event. This neurotoxicity can be both peripheral and central and can form a significant dose-limiting barrier in many clinical therapies (for example chemotherapies).

Cellular models have been developed to discover the processed behind these neurotoxic effects however they primarily rely on 2D monocultures of either primary animal or iPSC derived neuronal cells as their basis. Animal cell-based systems suffer from the inherent biological differences between species. Whilst humanised, iPSC-based monoculture systems, are often insufficient to discover the full pathology behind compound induced neurotoxicity due to their simplicity.

The present invention aims to ameliorate at least some of the problems associated with the models of the prior art by providing a more robust and physiologically relevant platform for assessing for example peripheral neuronal function in vitro.

### SUMMARY OF THE INVENTION

In a first aspect, provided herein is a cellular composite comprising a keratinocyte cell layer, a 3D (three dimensional) cell growth material, and a neuronal cell layer, wherein the 3D cell growth material is located between the keratinocyte cell layer and a neuronal cell layer, and wherein the 3D cell growth material compromises a mixed population of fibroblast cells and Schwann cells.

Suitably, the cellular composite may comprise a layer of a mixed population of fibroblast cells and Schwann cells between the keratinocyte cell layer and the 3D cell growth material.

Suitably, the Schwann cells are non-myelinating.

Suitably, the cellular composite may comprise neurites.

Suitably, the neurites may extend from the neuronal cell layer.

Suitably, the neurites may extend from the neuronal cell layer to the 3D cell growth material.

Suitably, the neurites may extend to the keratinocyte cell layer.

Suitably, the 3D cell growth material may be a porous scaffold or a gel.

Suitably, the porous scaffold may comprise or consist of a polymer.

Suitably, the polymer may be selected from the group consisting of polystyrene, Teflon®, polycarbonate, polyester, or acrylate, further optionally wherein the scaffold may be Alvetex®.

Suitably, the gel may be a hydrogel.

Suitably, the hydrogel may be selected from the group consisting of HydroMatrix™ Peptide Hydrogel, MaxGel™ Human ECM, Hystem® Stem Cell Culture, Geltrex®, or Matrigel™.

Suitably, the 3D cell growth material may be coated, for example with a cellular coating agent.

Suitably, the cellular coating agent may be solution, optionally the solution may comprise Axol Surebond XF®, Poly-D-Lysine and PBS.

Suitably, the keratinocyte cell layer may comprise keratinocyte cells.

Suitably, the keratinocyte cell layer may comprise a monolayer or a multilayer of keratinocyte cells.

Suitably the keratinocyte cells may be human keratinocyte cells, for example neonatal human epidermal keratinocyte (HEKn) cells.

Suitably, the neuronal cell layer may neuronal cells.

Suitably, the neuronal cells may be derived from induced pluripotent stem (iPS) cells.

Suitably, the iPS cells may be derived from fibroblast cells, optionally human fibroblasts (for example neonatal human dermal fibroblast (HDFn) cells).

Suitably, the neuronal cells may have or may be capable of having neurite outgrowth.

Suitably, the neuronal cell layer may comprise a monolayer or multilayer of neuronal cells.

Suitably the mixed population of fibroblast cells and Schwann cells comprises or consists of fibroblast cells and Schwann cells.

Suitably, the fibroblast cells and Schwann cells may be at a cell number ratio of from about 1:1 to about 20:1.

Suitably the mixed population of fibroblast cells and Schwann cells may be at a cell number ratio of from about 3:1 to about 10:1.

Suitably the mixed population of fibroblast cells and Schwann cells may be at a cell number ratio of about 7:1.

Suitably, the fibroblasts cells may be mammalian, for example human, canine, feline, equine, or bovine.

Suitably, the human fibroblasts are HDFn cells.

Suitably, the Schwann cells may be mammalian, for example human, canine, feline, equine, or bovine.

In a second aspect, provided herein is a method of making a cellular composite comprising a keratinocyte cell layer, a 3D (three dimensional) cell growth material, and a neuronal cell layer, wherein the 3D cell growth material is located between the keratinocyte cell layer and a neuronal cell layer, and wherein the 3D cell growth material compromises a mixed population of fibroblast cells and Schwann cells, wherein the method comprises the steps of:
a) seeding fibroblast cells and Schwann cells into the 3D cell growth material;
b) seeding neuronal cells on a first outer surface of the 3D cell growth material; and
c) seeding keratinocyte cells on a second outer surface of the 3D cell growth material, wherein the first outer surface and second surfaces are substantially parallel to one another and the 3D cell growth material is located between the first outer surface and the second outer surface.

Suitably, the method may comprise the step of coating the 3D cell growth material (for example with a cellular coating agent).

Suitably, the Schwann cells are non-myelinating.

Suitably, the step of coating the 3D growth material with a cellular coating agent may be prior to step b) of the method.

Suitably the cellular coating agent may be a solution, optionally the solution may comprise Axol Surebond XF®, Poly-D-Lysine and PBS.

Suitably, the fibroblast cells and Schwann cells may be seeded simultaneously or sequentially.

Suitably, the fibroblast cells and Schwann cells may be seeded at a cell number ratio of from about 1:1 to about 20:1.

Suitably, the fibroblast cells and Schwann cells may be seeded at a cell number ratio of from about 3:1 to about 10:1.

Suitably, the fibroblast cells and Schwann cells may be seeded at a cell number ratio of about 7:1.

Suitably the fibroblast cells and Schwann cells may be cultured prior to step b) of the method.

Suitably, the neuronal cells may be seeded in the presence of neuronal plating media.

Suitably, the neuronal plating media may comprise a Rho kinase inhibitor.

Suitably, the neuronal cells may be cultured prior to step c) of the method.

Suitably, the seeded neuronal cells may be cultured in the presence of sensory neuronal maintenance media.

Suitably, the Schwann cells and neuronal cells are seeded at a substantially the same cell number.

Suitably, the keratinocyte cells are seeded at a substantially the same or greater cell number than the fibroblasts.

Suitably, the HEKn cells are seeded in EpiLife® media.

Suitably, the seeded HEKn cells may be cultured (for example from about 1 to about 10 days) prior to being exposed to air liquid interface (ALI).

Suitably, the seeded HEKn cells may be cultured for about 3 days prior being exposed to air liquid interface (ALI).

In a further aspect, the present invention provides use of the cellular composite as described herein as an in vitro model of the skin.

In a third aspect, provided herein is a method of screening a test agent for pharmacological activity on cells, the method comprising:
a) providing a cellular composite as described herein;
b) exposing the cellular composite to the test agent; and
c) determining the phenotype of the cells of the cellular composite in response to the test agent, and thereby determining if the test agent has a pharmacological activity on the cells.

Suitably, the phenotype may be determined on neuronal cells, Schwann cells, fibroblasts, and/or keratinocytes.

Suitably, the Schwann cells are non-myelinating.

Suitably, the method may be a high throughput screening method.

In a fourth aspect, provided herein is use of the cellular composite of the first aspect as an in vitro model of the skin.

Suitably, the model may be of healthy or diseased skin.

In a fifth aspect, provided herein is use of the cellular composite according to the first aspect as an in vitro model of pain, optionally wherein the pain is neuropathic pain.

In further aspect, provided herein is an in vitro model for studying pain, or an in vitro model of the skin, optionally wherein the pain is neuropathic pain.

It will be appreciated that except for where the context requires otherwise, embodiments described with reference to one aspect of the invention may also be applied to other aspects of the invention. For example embodiments relating to the cellular composite also apply to the methods and uses of the invention, and vice versa.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Various aspects of the invention are described in further detail below.

### BRIEF DESCRIPTION OF THE DRAWINGS

Embodiments of the invention are further described hereinafter with reference to the accompanying drawings, in which:

FIG. 1A shows a schematic representation of the innervated 3D skin model. Quad co-culture 3D skin model consisting of an innervated dermis including neuronal, fibroblast and Schwann cell lineages directly cultured together within an inert Poly HIPE scaffold, Alvetex Scaffold®. Keratinocytes are cultured on the surface of the model to create a stratified epidermis replicating human epidermal structure. B shows H&E staining of full thickness innervated 3D Skimmune® model. Physiological tissue structures can be observed within the 3D constructed models. Keratinocytes are able to differentiate into a stratified epidermal layer consistent with normal human tissue morphology. Basal, granular and corneum layers can be clearly observed within the model. Dermal cells (fibroblasts and Schwann lineages) are cultured within the Alvetex Scaffold and induced to proliferate. This forms a dense layer of cultured cells on the surface of the polyHIPE, important for correct epidermal formation. Neuronal cells are grown on the base of the model and are induced to differentiate through the developing dermis by neurite extension to eventually interact with the basal keratinocytes.

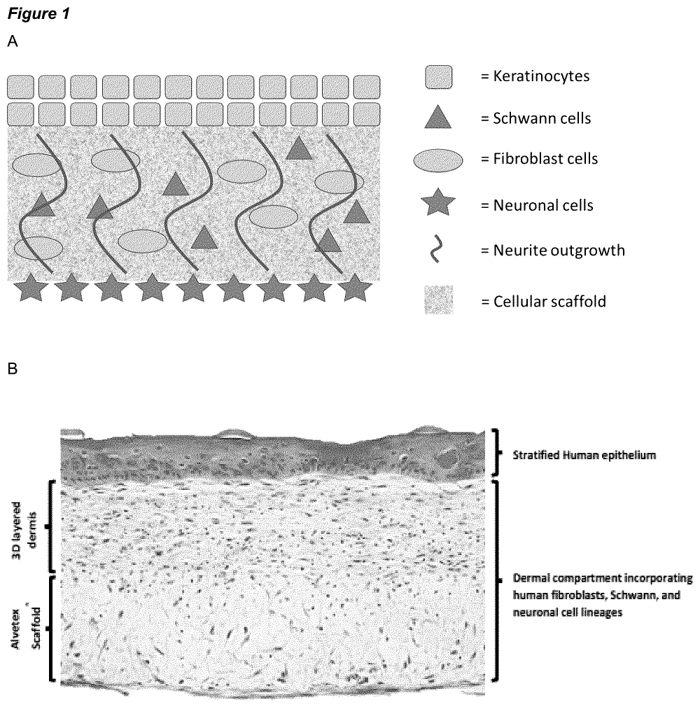

FIG. 2A shows TUJ1 staining of full thickness innervated 3D Skimune® model—TUJ1 staining specifically stains neuronal lineages within the 3D innervated Skimune® model. Neuronal cell bodies are primarily located on the base of the model. TUJ1 positive neurites can be observed transitioning through the dermal layer toward the apical surface of the model, eventually leading to epidermal innervation. White arrows highlight areas of clear neurite extension into the dermis. B shows high resolution imaging of neurites immediately beneath the epidermal layers—TUJ1 staining can be observed immediately beneath the epidermal layer, demonstrating neurite extension of >250 μM. White arrows highlight areas of clear neurite staining.

FIG. 3A Positive control Myelin stain of mouse brain tissue. B Positive staining for S100 (Noted by arrows) highlights areas with Schwann cells. Schwann cells appear to be co-localising with the neuronal cells present on the base of the model. C Notable absence of myelin staining co-localised with S100 staining and TUJ1 staining for neuronal cells. D TUJ1 staining on the base of the models demonstrated the localisation of the neuronal population within the scaffold.

### DETAILED DESCRIPTION

The present invention is based on the inventors' development of a novel cellular composite which may be used as a skin model. Advantageously, the skin model may support neurite outgrowth spanning the thickness of the composite, resulting in the development of an innervated skin model.

The cellular composite of the present invention has a number of advantageous properties as compared to other skin models in the art. One of the main advantages is that the cellular composite may be free of any xenobiotic components (such as for example collagen foam). This enables the cellular composite to more closely mimic the skin as it would be found in vivo.

Additionally, the present inventors believe that the cellular composite of the invention is particularly good at supporting neurite outgrowth due to the unique placement of the Schwann cells inside the 3D cell growth material, and optionally on an outer surface of the cell growth material. Without wishing to be bound by this hypothesis, the inventors believe that this positioning of the Schwann cells through a substantial thickness of the cellular composite enhances neurite outgrowth from the neuronal cells, as it enables the neurites to be in closer proximity with the neurites as they grow and extend through the layers of the cellular composite.

Furthermore, the cellular composite is simple to make, and can be adapted to be compatible with a 96-well plate rendering it especially useful in the context of personalised medicine. It will also be appreciated that the cellular composite may be made using autologous cells, rendering it even more useful in this context.

### A Cellular Composite

In one aspect, provided herein is a cellular composite comprising a keratinocyte cell layer, a 3D (three dimensional) cell growth material, and a neuronal cell layer, wherein the 3D cell growth material is located between the keratinocyte cell layer and a neuronal cell layer, and wherein the 3D cell growth material comprises a mixed population of fibroblast cells and Schwann cells.

The term “cellular composite” as used herein refers to an isolated artificial cell structure, i.e. not naturally occurring in the human or animal body. The cellular composite provides an ex vivo organ structure that closely represents in vivo organ structure. In the present case, the organ is the skin. In the context of the present disclosure, the cellular composite may be referred to as “the skin model”, “the 3D skin model”, or “the 3D Skimune® model”.

The cellular composite comprises a 3D cell growth material (also referred to herein as the cell growth material).

The “3D cell growth material” as used herein refers to a synthetic structure within which a mixed population of fibroblast cells and Schwann cells is located.

Suitably, the 3D cell growth material may be xenobiotic free. For the purposes of this disclosure, “xenobiotic free” (also referred to as xeno-free) means having no or substantially no xenogeneic products of non-human animal origin, such as cells, tissues, proteins, and/or body fluids, or any tissue or blood components, such as serum, which contain variable and undefined factors. Suitably, the 3D cell growth material is not a collagen foam.

The 3D cell growth material may support 3D culture of a mixed cell population of fibroblast cells and Schwann cells. By 3D culture it is meant that the cells of the cell population are able to adopt their natural 3D morphology and distribution within the cell growth material. Accordingly, the 3D cell growth material may provide a 3D support within which the fibroblast cells and Schwan cells are held such that the natural 3D morphology of the cells is maintained and such that the natural 3D distribution of cells is supported. The cells may proliferate in three dimensions within the 3D cell growth material and migrate within the 3D cell growth material to form an organisation of cells that mimics the in vivo skin cell arrangement.

Suitably, the mixed population of fibroblast cells and Schwann cells may proliferate and migrate to the outer surface of the 3D cell growth material and form a layer of a mixed population of fibroblast cells and Schwann cells on the 3D cell growth material. Accordingly, the cellular composite of the present invention may comprise a layer of a mixed population of fibroblast cells and Schwann cells on an outer layer of the 3D cell growth material. Suitably the layer of a mixed population of fibroblast cells and Schwann cells is between the keratinocyte cell layer and the 3D cell growth material.

In a suitable embodiment, the 3D cell growth material may be between about 10-1000 μm thick, or between about 50 to about 500 μm, or between about 100 to 300 μm. Suitably the 3D cell growth material (for example Alvetex) is about 200 μm, about 300 μm, about 400 μm, about 500 μm, about 600 μm, about 700 μm, about 800 μm, about 900 μm, or about 1000 μm thick.

Suitably, the 3D cell growth material may be formed from a scaffold or a gel.

In an embodiment where the 3D cell growth material is a gel, the gel may be a hydrogel.

Suitably, the gel may be selected from, for example, HydroMatrix™ Peptide Hydrogel, MaxGel™ Human ECM, Hystem® Stem Cell Culture, Geltrex®, or Matrigel™. Other suitable gels for culturing cells such as fibroblasts and/or Schwann cells will be known to those skilled in the art.

It will be appreciated that in an embodiment where the cell growth material is a gel (for example a hydrogel), the gel may encapsulate the mixed population of fibroblast cells and Schwann cells, and thereby comprise or consist of fibroblast cells and Schwann cells.

In an embodiment where the 3D cell growth material is a scaffold, the scaffold suitably comprises pores (i.e. is a porous scaffold). It will be appreciated that the pores shall be of a sufficient size to allow fibroblast cells and Schwan cells to infiltrate the pores. Thus suitably, the pores may be between about 10-100 μm, or between about 25-75 μm, or between about 30-40 μm. Suitably the pores may be between about 36-40 μm.

The scaffold may comprise a porosity of over 50%, over 60%, over 70%, over 80% or over 90%. In the context of the scaffold, the scaffold may be said to comprise a mixed population of fibroblast cells and Schwann cells when the cells are attached to the scaffold within the pores.

The scaffold may be formed from a polymer. The polymer may be any polymer suitable for cell culture. Suitable for cell culture polymers will be known to those skilled in the art. Merely by way of example, the polymer may be may be selected from, for example, polystyrene, Teflon®, polycarbonate, polyester, or acrylate. More suitably the scaffold is formed from polystyrene.

The scaffold may comprise a foamed material. Suitably the scaffold is a foamed polymer.

Suitably the scaffold is foamed polystyrene.

More suitably, the scaffold material is the Alvetex® material.

Suitably the polymer is inert (i.e. unable to substantially chemically interact with the cells cultures in or on the polymer).

The cell growth material is suitable to rest within a culturing vessel. Merely by way of example the cell culture vessel is a 96-well, 24-well, 12-well, or 6-well cell culture plate.

Suitably, the 3D cell growth material may be 3D printed.

Suitably, the 3D cell growth material may be coated with a cellular coating agent. The agent may be a solution, i.e. a cellular coating solution. A cellular coating agent is an agent that improves neuronal cell attachment and/or improves neurite outgrowth from the neuronal cells.

Suitably, the cellular coating solution may comprise Axol Surebond XF®, Poly-D-Lysine and PBS. Suitably, the cellular coating solution may comprise Axol Surebond XF® and PBS at a ratio of 1:200, and/or 0.1 mg/mL of Poly-D-Lysine.

Methods of a coating cell growth material will be well known to those skilled in the art. Merely by way of example, coating may be by submerging the cell growth material in the cellular coating solution, or by spraying the cellular coating solution onto the cell growth material.

As mentioned, the 3D cell growth material comprises a mixed population of fibroblast cells and Schwann cells. As used herein the term “mixed population” means that fibroblast cells and Schwann cells are randomly dispersed in the 3D cell growth material and the two types of cells (fibroblast cells and Schwann cells) are able to interact with each other.

The term “fibroblast” as used herein refers to a connective tissue cell that makes and secretes the extracellular matrix proteins, including, but not limited to, collagen. Fibroblasts, the most common cell type found in connective tissues, play an important role in healing wounds. Like other cells of connective tissue, fibroblasts are derived from primitive mesenchyme (a type of loose connective tissue derived from all three germ layers and located in the embryos).

Suitably, the fibroblasts cells are mammalian, for example human, canine, feline, equine, or bovine.

Suitably the fibroblast cell may be a human fibroblast cell, for example a neonatal human dermal fibroblast (HDFn) cell.

The term “Schwann cell”, as used herein, means a glial cell that in vivo has the ability to wrap around a nerve fiber in the peripheral nervous system (PNS), and forms the myelin sheaths of peripheral axons. In the PNS, Schwann cells play a role similar to that of oligodendrocytes in the central nervous system, providing myelination to PNS axons. Schwann cells in vivo also possess the capacity to present antigens to T-lymphocytes, and can be myelinating or non-myelinating.

Suitably, Schwann cells of the composite of the present invention may be preferably are non-myelinating. The present inventors believe that the use of non-myelinating Schwann cells results in a cellular composite that is a more physiologically relevant model of the skin and/or pain (such as neuropathic pain). This is because free nerve endings in the epidermis are not myelinated and are primarily responsible for nociception in the skin. The present inventors have shown that non-myelinating Schwann cells are capable of supporting neurite outgrowth in a physiologically relevant manner. Previously, it has been postulated that it is the myelin produced by Schwann cells that is responsible for promoting neurite outgrowth by forming myelin sheaths around neurites.

As used herein, the term non-myelinating Schwann cells refers to Schwann cells that substantially do not produce myelin. Methods for identifying non-myelinating Schwann cells will be known to those skilled in the art. As shown the Examples section of the present disclosure, non-myelinating Schwann may be identified by negative immunostaining with anti-myelin antibodies and/or positive immunostaining with antibodies selected from the group consisting of anti-S100 and anti-Beta-3 Tubulin.

Other methods for identifying non-myelinating Schwann cells will be also known to those skilled in the art. For example, non-myelinating Schwann cells may have comprise one or more markers selected from the group consisting of SOX10+, GAP43+, S100+, NCAM+, P75NTR+, EGR2−, MBP− and MPZ−. Suitably, non-myelinating Schwann cells may have comprise two, three, four, five, six, seven or all eight markers selected from SOX10+, GAP43+, S100+, NCAM+, P75NTR+, EGR2−, MBP− and MPZ−. Suitably, non-myelinating Schwann cells may be SOX10+, GAP43+, S100+, NCAM+, P75NTR+. Suitably, non-myelinating Schwann cells may be SOX10+, GAP43+, S100+, NCAM+, P75NTR+, EGR2−, MBP− and MPZ−. Methods of determining the presence or absence of these markers are well known in the art. Merely by way of example, the presence or absence of any one of these markers may be determined by a method such as flow cytometry or immunohistochemistry.

Suitably, the Schwann cell may be mammalian, for example human, canine, feline, equine, or bovine.

More suitably, the Schwann cell may be a human Schwann cell.

Suitably, the Schwann cell may be an iPSC-derived cell. It will be appreciated that the iPSC may be mammalian derived, i.e. derived from a cell of mammalian origin (for example from a human, canine, feline, equine, or bovine). Therefore, iPSC-derived Schwann cells may also be referred to as mammalian cells by virtue of mammalian origin of the iPSC.

Suitably, the fibroblast cells and Schwann cells may be in the 3D cell growth material at a cell number ratio of from about 1:1 to about 20:1. Suitably the mixed population of fibroblast cells and Schwann cells may be at a cell number ratio of from about 3:1 to about 10:1. Suitably the mixed population of fibroblast cells and Schwann cells is at a cell number ratio of about 7:1. For avoidance of doubt, when fibroblast cells and Schwann cells are at a ratio of about 7:1, this means that there is approximately seven times more fibroblast cells than Schwann cells.

In the context of the present disclosure, the term “about” or “approximately” mean within plus or minus 15% or less of the numerical value of the number with which it is being used. It shall be appreciated that the fibroblast cells and Schwann cells may be seeded in the 3D cell growth material at the rations mentioned above. However, as the cells attach to or become encapsulated by the 3D cell growth material and proliferate, the ratio of cells may be altered. Therefore the seeding ratio of the cells may not necessary be representative of the ratio of the cells in the cell composite of the invention. This may also be true in the context of neuronal cells and keratinocytes cells, as touched upon elsewhere in the present disclosure.

The term “neuronal cell layer” as used herein refers to a layer of cells, wherein the cells comprise or consist of neuronal cells. The neuronal cell layer may also be referred to as a “layer of neuronal cells”.

In some embodiments the neuronal cells may be primary neuronal cells, neuronal progenitor cells, or a mixture thereof.

Suitably, the cells may be seeded as neuronal progenitor cells and cultured to develop into mature neuronal cells. It will be appreciated that neuronal progenitor cells may develop into mature neuronal cells under specific culturing conditions. Such conditions will be well known to those skilled in the art, but merely by way of example may involve culturing the neuronal progenitor cells in neuronal maintenance media.

Suitably, the neuronal cell may be derived from induced pluripotent stem cells IPSCs.

Suitably, the iPSCs may be derived from fibroblast cells, optionally human fibroblasts (for example neonatal human dermal fibroblast (HDFn) cells).

The term “keratinocyte cell layer” as used herein refers to a layer of cells, wherein the cells comprise or consist of keratinocyte cells. The keratinocyte cell layer may also be referred to as a “layer of keratinocyte cells” or “layer of keratinocytes”.

“Keratinocyte” as used herein, refers to a skin cell of the keratinised layer of the epidermis.

Suitably, the keratinocyte cell may be mammalian, for example human, canine, feline, equine, or bovine.

More suitably the keratinocyte cell may be a human keratinocyte cell. For example, the human keratinocyte cell may be a neonatal human epidermal keratinocyte (HEKn) cell.

The cellular composite of the present invention has a layered structure. Suitably, the structure has at least three different layers. The term “different” in this context refers to the cellular composition of each of the three layers. The three different layers are the neuronal cell layer, the 3D cell growth material which comprises fibroblast cells and Schwann cells, and keratinocyte cell layer.

Suitably, the composite may comprise three or more layers, for example four or five layers.

More suitably, the cellular composite may comprise four layers. Suitably the fourth layer may be located between the 3D cell growth material comprising a mixed population of fibroblast cells and Schwann cells layer (also referred to herein as “the 3D cell growth material layer”) and the keratinocyte cell layer. Suitably the fourth layer may comprise a mixed population of fibroblast cells and Schwann cells layer.

It will be appreciated that such a fourth layer may be formed by the mixed population of cells initially seeded and cultured in the 3D cell growth material.

The keratinocyte cell layer and the neuronal cell layer, may be a monolayer or multilayer. The term “monolayer” shall be understood to mean an assembly of cells forming a sheet comprising no more than one cell in its thickness. The term “multilayer” shall be understood to mean an assembly of cells forming a sheet comprising more than one cell in its thickness. Suitably, the multilayer may comprise two, three, four, five, six, seven, eight, nine, ten, eleven, twelve, thirteen, fourteen, fifteen, sixteen, seventeen, eighteen, nineteen, twenty or more cells in its thickness.

Suitably, the neuronal cell layer may be a monolayer or multilayer. More suitably, the neuronal cell layer may be a monolayer.

Suitably, the keratinocyte cell layer may be a monolayer or a multilayer. More suitably, the keratinocyte cell layer may be a multilayer.

The term “monolayer” as used herein refers to a layer of cells one cell thick, grown in a culture. The term “multilayer” as used herein refers to a layer of cells more than one cell thick (for example two, three, four, five, six, seven, eight, nine, ten or more cells thick).

Suitably, the layer of a mixed population of fibroblast and Schwann cells located between the 3D cell growth material and the keratinocyte cell layer may be a multilayer.

Suitably, the cellular composite may comprise neurites. As used herein, the term “neurite” refers to any protrusion from a neuronal cell. Suitably, such a protrusion may be an axon and/or dendrite. It will be appreciated that when the composite comprises neurites, the neurites extend from neuronal cells in the neuronal cell layer.

As touched upon elsewhere in the present disclosure, the present inventors believe that the cellular composite of the present invention supports neurite outgrowth particularly well due to the presence of Schwann cells in the 3D cell growth material, and optionally in a layer between the 3D cell growth material and keratinocyte cells. This results in the Schwan cells being located across a significant portion of the thickness of the cellular composite. The thickness in this context refers to the distance from the outermost of the neuronal cell layer to the outermost of the keratinocyte cell layer. A significant portion of the thickness is at least about 50% of the distance defining the thickness.

The term “neurite outgrowth” refers to the extension of existing protrusion from a neuronal cell (e.g., axons and dendrites) and/or the growth or sprouting of new protrusion (e.g., axons and dendrites). Neurite outgrowth allows neural connectivity between the cells in the different layers of the cellular composite of the invention.

Suitably, the neurites may extend from the neuronal cell layer to the 3D cell growth material, and optionally to a mixed population of fibroblast cells and Schwann cells layer located between the 3D cell growth material and keratinocyte cell layer. In this context, when it is said that the neurites extend to a layer, it is meant that the neurites extend sufficiently to at least make contact with the layer they extend to.

Suitably, the neurites may extend from the neuronal cell layer to the keratinocyte cell layer.

Suitably, in the context of the present disclosure, the mixed population of fibroblast cells and Schwann cells layer located between the 3D cell growth material and keratinocyte cell layer may be referred to as the “dermal layer” or the “dermis”.

The keratinocyte cell layer may be referred to as the “epidermal layer” of the “epidermis”. Suitably, the neurite outgrowth may extend to immediately below the epidermis. Such neurite outgrowth may therefore innervate the epidermis.

In some embodiments, Schwann cells are optionally present in the 3D cell growth material comprises. In such embodiments the 3D cell growth material comprises or consists of a population of fibroblast cells. This embodiment gives rise to a further aspect of the invention providing a cellular composite comprising a keratinocyte cell layer, a 3D (three dimensional) cell growth material, and a neuronal cell layer, wherein the 3D cell growth material is located between the keratinocyte cell layer and a neuronal cell layer, and wherein the 3D cell growth material comprises a population of fibroblast cells.

### A Method of Making a Cellular Composite

In one aspect, the present invention provides a method of making a cellular composite comprising a keratinocyte cell layer, a 3D (three dimensional) cell growth material, and a neuronal cell layer, wherein the 3D cell growth material is located between the keratinocyte cell layer and a neuronal cell layer, and wherein the 3D cell growth material compromises a mixed population of fibroblast cells and Schwann cells, wherein the method comprises the step of:
a) seeding fibroblast cells and Schwann cells into the 3D cell growth material;
b) seeding neuronal cells on a first outer surface of the 3D cell growth material; and
c) seeding keratinocyte cells on a second outer surface of the 3D cell growth material, wherein the first outer surface and second surfaces are substantially parallel to one another and the 3D cell growth material is located between the first outer surface and the second outer surface.

The formation of the cellular composite of the present invention involves seeding of the cells which form the cellular composite, i.e. fibroblast cells, Schwann cells, neuronal cells, and keratinocyte cells.

Suitably, the mixed population of fibroblasts and/or Schwann cells may be seeded first.

Suitably, the fibroblast cells and Schwann cells may be seeded simultaneously or sequentially.

Suitably, the fibroblast cells and Schwann cells may be seeded at a cell number ratio of from about 1:1 to about 20:1.

Suitably, the fibroblast cells and Schwann cells may be seeded at a cell number ratio of from about 3:1 to about 10:1.

Suitably, the fibroblast cells and Schwann cells may be seeded at a cell number ratio of about 7:1.

Suitably, in the context of a 3D cell growth material compatible with a 96-well plate, the cell number ratio of about 7:1 may be about 350,000 fibroblasts to about 50,000 Schwann cells.

In the context of the fibroblast cells and/or Schwann cells, the terms “seeding” refers to bringing the cells into contact with the 3D cell growth material for a sufficient time such that they adhere to the 3D cell growth material or encapsulating the cells in the 3D cell growth material.

In an embodiment where the 3D culture material is a porous scaffold, the term seeding may refer to bringing the cells into contact with the 3D cell growth material for a sufficient time such that the cells adhere within the pores of the scaffold. Suitably, the 3D material may be coated with a cellular coating solution prior to bringing the fibroblast cells and/or Schwann cells into contact with it.

Suitably, the time sufficient for the fibroblast cells and/or Schwann cells to adhere or become encapsulated by the 3D cell growth material may be at least about 1 minute, at least about 5 minutes, at least about 10 minutes, at least about 30 minutes, at least about 1 hour, at least about 2 hours, at least about 3 hours, at least about 4 hours, at least about 5 hours, at least about 6 hours, at least about 7 hours, at least about 8 hours or more. Suitably in the context of a scaffold 3D cell growth material, the time sufficient for the fibroblast cells and/or Schwann cells to adhere may be about 2 hours, about 3 hours, about 4 hours, about 5 hours, about 6 hours, about 7 hours, about 8 hours or more. More suitably, the time may be about 4 hours.

Suitably, the fibroblast and/or Schwann cells may be seeded in DMEM medium. Suitably, the DMEM medium may contain an antibiotic (for example penicillin streptomycin (P/S), for example may contain 1% P/S), L-Glutamine (for example 2 mM) and FBS (for example 10%). Other types of media suitable for seeding and/or culturing fibroblast and/or Schwann cells will be known to those skilled in the art.

Suitably, after seeding, the fibroblast cells and/or Schwann cells may be cultured prior to seeding the neuronal cells and/or keratinocyte cells. The term “cultured” as used herein refers to one or more cells that are undergoing cell division, i.e. are proliferating.

The fibroblast cells and/or Schwann cells may be cultured in DMEM medium. Suitably, the DMEM medium may contain an antibiotic (for example penicillin streptomycin (P/S), for example may contain 1% P/S), L-Glutamine (for example 2 mM) and FBS (for example 10%).

Suitably the cells may be cultured for at least about 6 hours, at least about 12 hours, at least about 24 hours (1 day), at least about 2 days, at least about 3 days, at least about 4 days, at least about 5 days, at least about 6 days, at least about 7 days, at least about 8 days, or more. Suitably the fibroblast cells and/or Schwann cells may be cultured for about 4 days. Suitably the cells may be cultured until a layer of fibroblast cells and/or Schwann cells is formed on an outer surface of the 3D cell growth material. Suitably the layer formed on the outer surface of the 3D cell growth material is a layer of a mixed population of fibroblast cells and Schwann cells.

As mentioned, the 3D cell growth material comprises a first and second outer surface, and the 3D cell growth material located (i.e. sandwiched) between the first and second outer surface. In the context of the present disclosure the first outer surface is the surface that is closer to the neuronal cell layer and/or the surface on which the neuronal cells are seeded. This surface may also be referred to as the bottom surface, as in use (for example in a screening method as described herein below) it may be desirable to maintain the neuronal cell layer closer to the bottom of cell culture flask or plate. The second outer surface is the surface that is closer to the keratinocyte cell layer and/or the surface that the keratinocyte cells are seeded. Therefore, the outer surface of the 3D cell growth material on which the layer of fibroblast cells and/or Schwann cells is formed is the second out surface. The second surface may also be referred to as the top surface.

Thus, suitably, the method of making the cellular composite may comprise the steps of:
a) seeding fibroblast cells and Schwann cells into the 3D cell growth material;
b) culturing the fibroblast cells and Schwann cells in the 3D cell growth material;
c) seeding neuronal cells on a first outer surface of the 3D cell growth material; and
d) seeding keratinocyte cells on a second outer surface of the 3D cell growth material, wherein the first outer surface and second surfaces are substantially parallel to one another and the 3D cell growth material is located between the first outer surface and the second outer surface.

Suitably, the fibroblast cells and Schwann cells are cultured until a layer of a mixed population of fibroblast cells and Schwann cells is formed on a second outer surface of the 3D cell growth material, prior to seeding the keratinocyte cells.

In the context of “neuronal cells” the term “seeding” refers to bringing the neuronal cells into contact with the 3D cell growth material for a sufficient time such that the cell adhere to a first (bottom) outer layer of the 3D cell growth material. Suitably, the 3D cell growth may be coated with a cellular coating agent.

It will be appreciated that in order to seed the neuronal cells the 3D cell growth material may be inverted, such that the first outer layer is above the second outer layer.

Suitably, the time sufficient for the neuronal cells to adhere to the 3D cell growth material may be at least about 1 minute, at least about 5 minutes, at least about 10 minutes, at least about 30 minutes, at least about 1 hour, at least about 2 hours, at least about 3 hours, at least about 4 hours, at least about 5 hours, at least about 6 hours, at least about 7 hours, at least about 8 hours or more. Suitably time sufficient for neuronal cells to adhere may be about 2 hours, about 3 hours, about 4 hours, about 5 hours, about 6 hours, about 7 hours, about 8 hours or more. More suitably, the time may be about 2 hours.

Suitably the neuronal cells being seeded may be neuronal progenitor cells (NPCs).

Suitably, the number or neuronal cells (e.g. NPCs) seeded is the about the same as the number of seeded Schwann cells. For example, in the context of a 3D cell growth material that is compatible with a 96-well plate, approximately 50,000 neuronal cells (e.g. NPCs) are seeded.

Suitably, the neuronal cells are seeded in neuronal plating medium. Suitably the neuronal plating medium may be supplemented with a Rho kinase inhibitor.

Suitably, after seeding the neuronal cells may be cultured. Suitably the cells may be cultured for at least about 1, at least about 2, at least about 3, at least about 4, at least about 5, at least about 6, at least about 7, at least about 18, at least about 9, at least about 10, or more days. Suitably, the neuronal cells may be cultured for at least about 11, at least about 12, at least about 13, at least about 14, at least about 15, at least about 16, about 17, at least about 18, at least about 19, at least about 20, at least about 21, at least about 22 at least, at least about 23, at least about 24, at least about 25, at least about 26, at least about 27, at least about 28, at least about 29, at least about 30, or more days. Suitably, the neuronal cells are cultured from about 15 to about 25 days, more suitably about 21 days.

The neuronal cells may be cultured in sensory neuronal maintenance media. Suitably the sensory neuronal maintenance media may comprise 10 μg/mL GDNF, 10 μg/mL NGF, 10 μg/mL BDNF, 10 μg/mL NT-3 and 1% (v/v) sensory maturation maximiser supplement as explained in more detail in the examples section below. Other types of sensory neuronal maintenance media will be known to those skilled in the art. It will also be appreciated by those skilled in the art that it may be desirable to change the type of media used depending on cell type (for example if a different neuronal lineage is used).

Suitably at about day 2, about day 3, about day 4, about day 5, or about day 6, the neuronal cells may be treated with Mitomycin C (for example about 2.5 μg/mL Mitomycin C). In this context, as it will be appreciated by a person skilled in the art, the term “treated” may mean that Mitomycin C is added to the media in which the cells are cultured. The treatment may be for about 30 minutes, about 1 hours, about 2 hours, about 3 hours, about 4 hours. Suitably it may be for about 2 hours. The treatment may be stopped by placing the 3D cell growth material into fresh media (without the Mitomycin C).

Thus, suitably, the method of making the cellular composite of the invention may comprise the steps of:
a) seeding fibroblast cells and Schwann cells into the 3D cell growth material;
b) culturing the fibroblast cells and Schwann cells in the 3D cell growth material;
c) seeding neuronal cells (for example neuronal progenitor cells) on a first outer surface of the 3D cell growth material;
d) culturing the neuronal cells; and
e) seeding keratinocyte cells on a second outer surface of the 3D cell growth material, wherein the first outer surface and second surface are substantially parallel to one another and the 3D cell growth material is located between the first outer surface and the second outer surface.

Suitably, the fibroblast cells and Schwann cells may be cultured until a layer of a mixed population of fibroblast cells and Schwann cells is formed on a second outer surface of the 3D cell growth material, prior to seeding the keratinocyte cells.

Thus, in the context of keratinocyte cells, the term “seeding” may refer to bringing the cells in directly or indirectly with the second outer surface of the 3D cell growth material for a sufficient time such that the cell adhere directly or indirectly to the second outer surface of the 3D cell growth material. As mentioned above, the fibroblast cells and Schwann cells may be cultured until a layer of a mixed population of fibroblast cells and Schwann cells is formed on a second outer surface of the 3D cell growth material, prior to seeding the keratinocyte cells. Therefore, when such a layer is formed, the keratinocyte cells may be said to be seeded indirectly on the second outer surface. In other words, they may be seeded on a layer comprising a mixed population of fibroblast cells and Schwann cells. By the same token, when such a layer is not present, and the keratinocyte cells are brought into contact with the 3D cell growth material. It such an embodiment, the keratinocytes are seeded directly onto the second surface.

Suitably, the keratinocyte cells may be at passage from 2 to 10 (for example 2 to 5, for example 2 or 3) at the time of seeding.

Suitably, the number of keratinocytes seeded is the about the same or greater than the number of seeded fibroblasts.

For example, in the context of a 3D cell growth material that is compatible with a 96-well plate, approximately 300,000 to 500,000 keratinocytes are seeded. More suitably, about 400,000 keratinocytes are seeded.

It will be appreciated that the step of seeding keratinocyte cells may involve inverting the 3D cell growth material such that the neuronal cell layer is at the bottom of the 3D cell growth material and the second outer surface is at the top.

Suitably the keratinocyte cells are seeded in EpiLife® media. Merely by way of example the EpiLife® media may comprise 1% (v/v) HKGS, 140 μM CaCl, 10 ng/mL KGF and 50 μg/mL Ascorbic acid, as explained in more detail in the examples section of the present disclosure. Other types of media suitable for seeding and/or culturing keratinocyte cells will be known to those skilled in the art.

Suitably, after seeding the keratinocyte cells may be cultured. Suitably, the keratinocyte may by cultured in the EpiLife® media. Suitably, the keratinocyte cells may be cultured for at least about 0.5 day, at least about 1 day, at least about 2 days, at least about 3 days, at least about 4 days, or at least about 5 days, or more, whilst fully submerged in the culture medium. Suitably, the keratinocyte cells may be cultured for about 3 days whilst fully submerged in the culture medium.

The cells (specifically the keratinocyte cells) may be then raised to air liquid interface and maintained at the air liquid interface for at least about 5, at least about 6, at least about 7, at least about 8, at least about 9, at least about 10, at least about 11, at least about 12, at least about 13, at least about 14, at least about 15, or more days. Suitably the keratinocyte cells may be raised to air liquid interface and maintained at the air liquid interface for at least about 10 days. Methods of culturing cells in the air liquid interface will be well known to those skilled in the art. Methods of culturing cells on and/or in 3D cell culture materials at the air liquid interface will also be well known to those skilled in the art. Merely by way of example, when the 3D scaffold is an Alvetex™ scaffold, the scaffold may be placed on a cell culture well or plate insert that enables the scaffold to be cultured without direct contact with the bottom surface of the cell culture well or plate.

In a further aspect, provided herein a cellular composite obtained by the method as described above.

### Methods of Screening

In a further aspect, the present invention provides a method of screening a test agent for pharmacological activity on cells, the method comprising:
a) providing a cellular composite as described herein;
b) exposing the cellular composite to the test agent; and
c) determining the phenotype of the cells of the cellular composite in response to the test agent, and thereby determining if the test agent has a pharmacological activity on the cells.

Suitably, when a test agent alters the phenotype of a cell exposed to said test agent, it can be said that the test agent has pharmacological activity on that cell. By the same token, if a test agent does not alter the phenotype of a cell exposed to the test agent, it can be said that the test agent does not have pharmacological activity on that cell.

It will be appreciated that depending on the type and/or purpose of the test agent, the phenotype may be determined on different types of cells within the cellular composite. Suitably, the cells may be neuronal cells, Schwann cells, fibroblasts, and/or keratinocytes.

By way of example, if the screening method is employed in order to determine whether a test agent has a pharmacological effect on neuronal cells (i.e. is for example neurotoxic, or neuroprotective) step c) may involve determining the phenotype of neuronal and/or Schwann cells of the cellular composite.

On the other hand, if the screening method is employed in order to determine whether a test agent has a pharmacological effect on skin, step c) may involve determining the phenotype of fibroblast and/or keratinocyte cells of the cellular composite.

The term “pharmacological activity” as used herein means an activity that modulates or alters biological processes to cause phenotypic changes (e.g., cell death, increase or decrease in cell proliferation, increase or decrease in neurite outgrowth, alteration in protein expression, alteration in cell activity, etc.) of the cells. Depending on the phenotypic changes and/or desired effect of the test agent on the cells, the pharmacological activity of the test agent may be positive or negative. It will be appreciated that in some circumstances a specific phenotypic change may be seen as positive, whereas in other circumstances a similar or same change may be, in fact, seen as negative. It will be appreciated that it is within the skilled persons knowledge to determine whether a test agent has a pharmacological activity on the cells of the composite, and/or whether said pharmacological activity is positive or negative. The skilled person will also appreciate that the change in phenotype indicative of pharmacological activity will be different depending on cell type and/or test agent. However, the skilled person will be well aware of methods, assays, and/or possible phenotypic changes that would allow to determine whether the test agent has a pharmacological effect. Exemplary phenotypic changes are discussed below.

In the context of neuronal cells, the phenotypic change indicative of pharmacological activity may be by way of example and not limitation an alteration to neuronal cell function, alteration to neuronal cell viability, alteration to neuronal cell proliferation, alteration to neurite outgrowth, and/or alteration to neuronal cell activity.

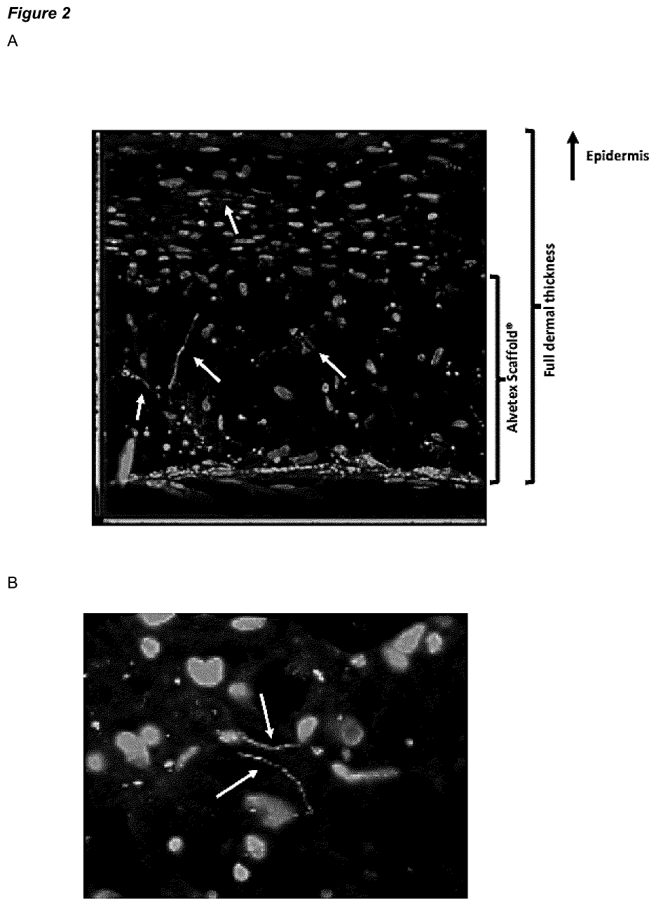

Methods of analysing neuronal cell phenotypes will be well known to those skilled in the art. Merely by way of example, neuronal cell viability may be determined through the use of a cell-permeable viability indicator dye that is converted by living cells to emit green fluorescence (excitation/emission ˜495 nm/˜515 nm). Neurite outgrowth may be determined for example by measuring neurite length, thickness and/or number of neurites per neuronal cell. Neuronal cell proliferation may be determined by measuring the expression of TUJ1. Methods of determining TUJ1 expression will be known to those with skill in the art. An exemplary method is also provided in the Examples section of the present disclosure. “TUJ1” as used herein refers to Beta-3 Tubulin which is a 50 KDa protein, a component of tubulin and a major component of the neuronal microtubule cytoskeleton. TUJ1 is known to be highly neuron specific and is widely used throughout the literature to identify neuronal cells from other distinct cell types. As shown in FIG. 2, the neuronal cells in the cellular composite of the present invention express TUJ1. Increased amounts of TUJ1 as compared to a suitable control may be indicative of increased neuronal cell number, proliferation and/or viability. Neuronal cell activity may be determined by measuring neuronal cell membrane potential and/or neuronal cell chemical (such as neurotransmitter for example substance P, calcitonin gene-related peptide, norepinephrine, noradrenalin, neuropeptide Y, and/or nerve growth factor) release.

As mentioned, whether the pharmacological activity of the test agent is positive or negative may depend on the desired aim of the test agent. For example, when the screening method is employed to identify pain relief agents, a reduction in neuronal cell activity (for example reduction in cell membrane potential, changes in neural transmitter release and/or changes in oxygen metabolic rates) as compared to a suitable control may be seen as positive pharmacological activity. However, when the screening method is employed to identify a potential wound healing agent, the increase in neuronal cell activity (for example increased release of substance P and/or calcitonin-gene related peptide (CGRP) from the neuronal cells) may be seen as a positive pharmacological activity. The skilled person will understand when a pharmacological activity is positive or negative depending on the aim of the test agent.

Suitably, a test agent may be said to have a positive pharmacological activity when exposure of the cellular composite to the test agent results in improved neuronal cell phenotype (for example improved function, viability, proliferation, activity) as compared to a control. The control may be a cellular composite that has not been exposed to said test agent. The term “exposed” as used in the present disclosure means brought into contact. The cellular composite of the invention may be brought into contact with the test agent by incubating for a period of time (for example several minutes, hours or days) a part or all of the cellular composite with a medium that comprises the test agent.

Suitably, the test agent may have a protective effect and/or therapeutic effect on neuronal cell phenotype (for example increase function, increase viability, increase proliferation, and/or decrease activity). Suitably, the test agent may have a protective and/or therapeutic effect on neuronal cell function when used in combination with a compound known to have a negative effect on neuronal cell phenotype (for example decrease neuronal cell function, viability, proliferation, and/or cause abnormal activity). Such a compound known to have a negative effect in neuronal cell phenotype may be for example a neuropathy causing chemotherapeutic.

In such an embodiment, the protective and/or therapeutic effect may be determined as compared to a control which may be a cellular composite of the invention exposed to a compound known to have negative effect on neuronal cell phenotype and not exposed to the test agent. In this context, “in combination” need not necessarily mean “at the same time”, but may mean “at the same time”, “prior to”, and/or “after” exposure to a compound known to have such a negative pharmacological activity. When the test agent is provided at the same time or prior to exposure to a compound known to have a negative pharmacological activity on neuronal cells and the test agent improves neuronal cell phenotype it can be said that the test agent has a protective effect on neuronal cells. When the test agent is provided after exposure to a compound known to have a negative pharmacological activity on neuronal cells and the test agent improves neuronal cell phenotype (e.g. reverses fully or partially the negative effect of the compound known to have a negative pharmacological activity) it can be said that the test agent has a therapeutic effect on neuronal cells.

By the same token, a test agent may be said to have a negative pharmacological activity when the test agent adversely impacts neuronal cell phenotype. Adversely impacted neuronal cell phenotype may include reduced neuronal cell function, reduced neuronal cell viability, reduced neuronal cell proliferation, and/or abnormal neuronal cell activity, as compared to a control. In the connect of the present disclosure the terms “decreased” and “reduced” may be used interchangeably.

Suitably, the test agent that has a negative pharmacological activity on neuronal cells may be referred to as a neurotoxic or neuroirritant agent. A neurotoxic agent is an agent that reduces neuronal cell viability and/or proliferation. A neuroirritant agent is an agent that causes unwanted increased neuronal cell activation. Such an unwanted neuronal cell activation may be referred to as overactivation.

Merely by way of example and not limitation, the screening method of the invention may be used to identify an agent that is neurotoxic, neuroirritant, has wound healing properties, and/or relieves pain. Accordingly, it can be said that the screening method provided herein is a method of screening for a neurotoxic agent, neuroirritant agent, a wound healing agent, and/or pain relieving agent, respectively.

A pain relieving agent may be an agent that reduces neuronal cell activation and/or activity. Merely by way of example, in the screening method of the invention the neuronal cells of the cellular composite may be activated by capsaicin in order to then determine whether a test agent reduces neuronal activation.

Accordingly, the screening method of the invention may comprise the step of exposing the cellular composite to a neuronal cell activator (such as capsaicin). Suitably, the step of exposing the cellular composite to a neuronal cell activator (such as capsaicin) may be prior to or during step b) of the method.

As used herein, a “wound healing agent” is a compound or composition that promotes wound healing process. Wound healing process may be promoted by, for example, an increased release of substance P from neuronal cells and/or calcitonin-gene related peptide (CGRP). Thus a test agent that is a wound healing agent may result in substance P and/or calcitonin-gene related peptide (CGRP) release from neuronal cells exposed to said test agent.

Suitably, the cells in the cellular composite may be comprise or consist of certain autologous cells. Suitably, in the cellular composite the autologous cells may be the fibroblast and/or keratinocyte cells. Suitably, the neuronal cells and Schwann cells may be iPSC-derived. It will be appreciated that the use of autologous cells may be particularly beneficial in the context of personalised medicine for screening whether the test agent may have a pharmacological activity on cells in a subject from whom the autologous cells may be derived from.

Suitability, before step b) of the screening method is performed, the cellular composite may be washed (with saline, for phosphate buffered saline) in order to remove any xenobiotic components (for example FBS). It will be appreciated that such xenobiotic components may impact the results of the screening method, and therefore their removal may be highly advantageous in order to better mimic in vivo conditions.

Suitably, the screening methods of the invention may be a high throughput screening method.

The cellular composite of the invention may be exposed to a disease inducing compound prior to or during step b) of the screening method. Exposure to such a disease inducing compound may allow the cellular composite to model a disease state. Thus, a disease inducing compound may be a compound known to induce certain phenotypical changes associated with a diseases. It will be appreciated that the skilled person will know which disease inducing compound may be used in order to cause phenotypical changes associated with a disease. Merely by way of example, prior to exposing the cellular composite to the test agent, the cellular composite may be exposed to a disease inducing compound such as signalling proteins or peptides (for example, cytokines IL-6, IL-8 and TNF-α). These compounds may result in changes in neuronal distribution and neuronal activity, rendering the cellular composite a useful model for certain diseases (such as atopic dermatitis which is characterised by changes in neuronal distribution and activity within the skin). Accordingly, in a further aspect, the present invention provides use of the cellular composite as described herein as an in vitro model of the skin. Suitably, the model may be of healthy or diseased skin. It will be appreciated that in order for the cellular composite to be used as model of diseases skin (for example atopic dermatitis) the cellular composite may need to be exposed to a disease causing compound.

Suitably, model of healthy skin may be useful for example to study wound healing. The inventors believe that the cellular composite of the invention may be particularly useful in the context of wound healing because it is known that neuronal activation and/or neuronal chemical release may influence keratinocyte proliferation and migration, which occurs during wound healing.

### EXAMPLES

### Materials and methods

### 3D Model Step One—Dermal Schwann Cell Co-Culture

96 well Alvetex® Scaffolds were prepared for culture by complete immersion in 70% (v/v) ethanol for a minimum of 15 minutes with gentle agitation. Scaffold were then washed twice for 5 minutes in sterile PBS.

Cellular coating solution was created by diluting Surebond XF (AXOL, Cat#: ax0053) 1:200 and Poly-D-Lysine (Sigma, Cat#: P6407) to a final concentration of 0.1 mg/mL in sterile PBS. Alvetex® Scaffolds were placed within a 96 well plate and completely submerged in cellular coating solution (250 μL) for 4 hours at 37° C.

HDFn and Schwann cells were passaged and counted. 350,000 HDFn and 50,000 Schwann cells were seeded in 30μL of supplemented DMEM containing 1% P/S, 2 mM L-Glutamine and 10% FBS into the apical compartment of Alvetex Scaffold inserts.

Cells were allowed to attach for 2 hours at 37° C. before being completely submerged in 250 μL of supplemented DMEM. Models were media changed on alternate days for 4 days.

### Neuronal Culture

Neuronal plating medium (Axol, Cat#: ax0033) was removed from −80° C. storage and allowed to thaw overnight at 4° C. Neuronal plating media was supplemented with 10 ug/mL Y-27632 ROCK inhibitor (Sigma, Cat#: SCM075) before use for neuronal cell revival. Dermal/Schwann cell 3D co-cultures were incubated for 4 hours at 37° C. in DMEM supplemented with Surebond XF, 1:200 and Poly-D-Lysine to a final concentration of 0.1 mg/mL prior to neuronal cell revival.

Neuronal progenitor cells (Axol, Cat#: ax0055) were revived and counted as per the manufacturer's instructions. Coated dermal/Schwann cell models were washed once in PBS before being inverted and placed within a sterile Petri dish. This exposes the bottom of the model for cell seeding.

Neuronal progenitor cells were seeded at a density of 50,000 cells per model in 15 μL of ROCK inhibitor supplemented neuronal plating medium on the bottom of 3D co-cultures. Neuronal cells were allowed to attach for 2 hours at 37° C. After, models were moved back into 96 well plates and completely submerged overnight in supplemented neuronal plating media.

The following day models were switched into sensory neuronal maintenance media (AXOL, Cat#: ax0060) supplemented with 10 μg/mL GDNF (AXOL, Cat#: ax139855), 10 μg/mL NGF (AXOL, Cat#: ax139789), 10 μg/mL BDNF (AXOL, Cat#: 139800), 10 μg/mL NT-3 (AXOL, Cat#: ax139811) and 1% (v/v) Sensory maturation maximiser supplement (AXOL, Cat#: ax0058).

On day 4 post neuronal seeding models were treated with 2.5 μg/mL Mitomycin C (Sigma, Cat#: M4287) in sensory maintenance media for 2 hours at 37° C. After mitomycin C treatment models were washed twice in PBS before place back into fully supplemented sensory maintenance media. Models were media changed 50% every 2 days until 21 days post neuronal seeding.

### Epidermal Cell Seeding

HEKn cells were expanded, passaged and counted. Neuronal 3D co-cultures were washed 3 times in PBS to remove all sensory maintenance media.

HEKn cells were seeded into the apical compartment of 3D co-culture models at a density of 400,000 cells per model in 30 μL EpiLife supplemented with 1% (v/v) HKGS, 140 μM CaCl (Sigma, Cat#: C5670), 10 ng/ml KGF (Peprotech, Cat#: 100-19) and 50 μg/mL Ascorbic acid (Sigma, Cat#: A92902). HEKn cells were allowed to attach for 2 hours before models were moved into Alvetex® 96 well deep-dish plates and fully submerged in 1.5 mL supplemented EpiLife per model.

Day 3 after HEKn addition models were raised to air liquid interface (ALI) by complete aspiration of culture media and addition of 1 mL per model of EpiLife supplemented with 1% (v/v) HKGS, 1.64 mM CaCl and 50 μg/ml Ascorbic acid. Models were maintained at ALI for 10 days with media changes on alternate days.

### TuJ1 Staining of 3D Neuronal Skin Models

Neuronal skin models were fixed in formalin overnight at 4° C. before being dehydrated and processed into paraffin wax. 7 μm sections were taken of the neuronal skin models and heat fixed to glass microscope slides at 60° C. for 2 hours. Prior to staining, samples on glass microscope slides were deparaffinised in xylene and rehydrated through ethanol gradient and brought to water. Samples were blocked in a 10% normal goat serum (NGS) in PBS for 1 hour at room temperature. TuJ1 antibody (SantaCruz, sc-80005) was diluted 1:200 in PBS. Samples were incubated in antibody solution for 2 hours at room temperature. After, samples were washed and incubated with anti-goat secondary antibody conjugated with Alexa® 488 for 1 hours at room temperature. Samples were washed once again before mounting in Fluoroshield containing DAPI. Samples were imaged on a Zeiss 800 confocal microscope.

### Myelin, S100 and Beta-3 Tubulin Staining

Sections were cut to 4 μm thickness and baked at 60° C. for 2 hours. Sections were then cleared and taken to water then incubated with heated citrate buffer for heat mediated antigen retrieval.

Following antigen retrieval sections were permeabilised using 0.2% triton x. 10% goat serum was then used to block the sections for 30 minutes.

After 30 minutes the samples were incubated with primary antibodies at room temperature for 1 hour. The primary antibodies used were:
Rabbit anti-myelin mAb used at 1:100.
Mouse anti-s100 used at 1:100.
Mouse anti-tubulin beta 3 used at 1:100.

Excess primary antibodies were removed with wash buffer and slides were incubated with secondary antibodies.

The secondary antibodies used were:
Goat anti-mouse Alexa Fluor 488 conjugated used at 1:400
Goat anti-rabbit Alexa Fluor 647 conjugated used at 1:400

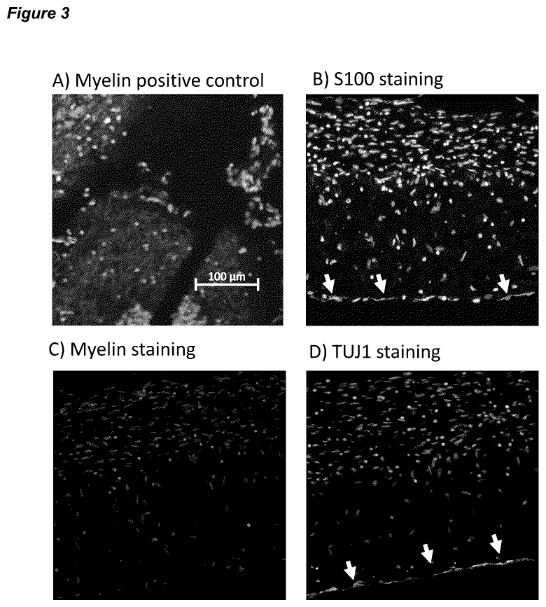

After the final wash to remove excess secondary antibodies, coverslips were fixed to slides using mounting medium with DAPI. Results are seen in FIG. 3.

1. A cellular composite comprising:
a keratinocyte cell layer;
a 3D (three dimensional) cell growth material; and
a neuronal cell layer,
wherein the 3D cell growth material is located between the keratinocyte cell layer and the neuronal cell layer, and
wherein the 3D cell growth material comprises a mixed population of fibroblast cells and Schwann cells.

2. The cellular composite of claim 1, wherein the cellular composite comprises a layer of the mixed population of fibroblast cells and Schwann cells between the keratinocyte cell layer and the 3D cell growth material.

3. The cellular composite of claim 1, wherein the Schwann cells are non-myelinating.

4. The cellular composite of claim 1, wherein the cellular composite comprises neurites.

5. The cellular composite of claim 4, wherein the neurites extend from the neuronal cell layer.

6. The cellular composite of claim 1, wherein the 3D cell growth material is a porous scaffold or a gel.

7. The cellular composite of claim 6, wherein the porous scaffold comprises a polymer.

8. The cellular composite of claim 6, wherein the gel is a hydrogel.

9. The cellular composite of claim 1, wherein the 3D cell growth material is coated.

10. The cellular composite of claim 9, wherein the cellular coating agent is a solution.

11. The cellular composite of claim 1, wherein the keratinocyte cell layer comprises a monolayer or a multilayer of keratinocyte cells.

12. The cellular composite of claim 1, wherein the keratinocyte cells are mammalian keratinocyte cells.

13. The cellular composite of claim 1, wherein neuronal cells are derived from induced pluripotent stem (iPS) cells.

14. The cellular composite of claim 13, wherein the iPS cells are derived from mammalian cells.

15. (canceled)

16. The cellular composite of claim 1, wherein the neuronal cell layer comprises a monolayer or multilayer of neuronal cells.

17. The cellular composite of claim 1, wherein the mixed population of fibroblast cells and Schwann cells comprises fibroblast cells and Schwann cells at a cell number ratio of from about 1:1 to about 20:1.

18. The cellular composite of claim 1, wherein the fibroblasts cells are mammalian fibroblasts.

19. The cellular composite of claim 1, wherein the Schwann cells are mammalian Schwann cells.

20. A method of making a cellular composite comprising a keratinocyte cell layer, a 3D (three dimensional) cell growth material, and a neuronal cell layer, wherein the 3D cell growth material is located between the keratinocyte cell layer and the neuronal cell layer, and wherein the 3D cell growth material comprises a mixed population of fibroblast cells and Schwann cells, wherein the method comprises the steps of:
a) seeding fibroblast cells and Schwann cells into the 3D cell growth material;
b) seeding neuronal cells on a first outer surface of the 3D cell growth material; and
c) seeding keratinocyte cells on a second outer surface of the 3D cell growth material,
wherein the first outer surface and the second outer surface are substantially parallel to one another and the 3D cell growth material is located between the first outer surface and the second outer surface.

21-34. (canceled)

35. A method of screening a test agent for pharmacological activity on cells, the method comprising:
a) providing the cellular composite of claim 1;
b) exposing the cellular composite to the test agent; and
c) determining the phenotype of the cells of the cellular composite in response to the test agent, and thereby determining if the test agent has a pharmacological activity on the cells.

36-46. (canceled)
